(19) United States
(12) Patent Application Publication
(10) Pub. No.: US 2025/0299900 A1
(43) Pub. Date: Sep. 25, 2025

(54) X-RAY TUBE CONTROL SYSTEM AND X-RAY COMPUTED TOMOGRAPHY IMAGING APPARATUS

(71) Applicant: Canon Medical Systems Corporation, Otawara-shi (JP)

(72) Inventor: Masaharu TSUYUKI, Nasushiobara (JP)

(73) Assignee: Canon Medical Systems Corporation, Otawara-shi (JP)

(21) Appl. No.: 19/082,591

(22) Filed: Mar. 18, 2025

(30) Foreign Application Priority Data
Mar. 22, 2024 (JP) 2024-047139

(51) Int. Cl. H01J 35/02; G01T 1/29; H01J 35/04

(52) U.S. Cl. CPC H01J 35/025; G01T 1/2985; H01J 35/045

(57) ABSTRACT

According to one embodiment, an X-ray tube control system includes a tube voltage control circuitry and a grid control circuitry. The grid control circuitry performs control to decrease a grid voltage to a first (ex. low) grid voltage and control to hold the first grid voltage exclusively within a first transition period in which a tube voltage makes a transition from a second (ex. high) tube voltage to a first tube voltage and a first (ex. low) hold period in which a tube voltage is held at the first tube voltage so as to prevent a tube current from exceeding an upper limit tube current based on allowable power.

### CROSS-REFERENCE TO RELATED APPLICATIONS

This application is based upon and claims the benefit of priority from Japanese Patent Application No. 2024-047139, filed Mar. 22, 2024, the entire contents of which are incorporated herein by reference

### FIELD

Embodiments described herein relate generally to an X-ray tube control system and an X-ray computed tomography imaging apparatus.

### BACKGROUND

There is available dual energy scan, which is designed to perform X-ray CT (Computed Tomography) imaging while alternately switching a tube voltage between a high tube voltage and a low tube voltage. In dual energy scan, in order to homogenize the quality of projection data acquired upon the application of a high tube voltage and of projection data acquired upon the application of a low tube voltage, it is desired to acquire projection data with the same X-ray dose at the time of application of the high tube voltage and at the time of application of the low tube voltage.

### BRIEF DESCRIPTION OF THE DRAWINGS

FIG. 1 is a view showing an example of the arrangement of an X-ray computed tomography imaging apparatus according to the present embodiment.

FIG. 2 is a block diagram showing an example of the arrangement of an X-ray tube control system according to the present embodiment.

FIG. 3 is a timing chart of kV switching in Comparative Example 1.

FIG. 4 is a timing chart of kV switching in Comparative Example 2.

FIG. 5 is a timing chart of kV switching in Example 1.

FIG. 6 is a timing chart of kV switching in Example 2.

FIG. 7 is a block diagram showing an example of the arrangement of a grid power supply circuitry according to Example 2.

FIG. 8 is a schematic circuitry diagram of a grid power supply circuitry for both series connection and parallel connection, showing the connection relationship between switches forming a series circuitry at the time of application of a high voltage.

FIG. 9 is a schematic circuitry diagram of a grid power supply circuitry for both series connection and parallel connection, showing the connection relationship between switches forming a parallel circuitry at the time of application of a low voltage.

FIG. 10 is a timing chart of kV switching in Example 3.

FIG. 11 is a timing chart of kV switching in Example 4.

FIG. 12 is a timing chart of kV switching in Example 5.

### DETAILED DESCRIPTION

An X-ray tube control system according to an embodiment includes a tube voltage control unit and a grid control unit. The tube voltage control unit alternately switches the tube voltage applied between a cathode that emits electrons and an anode that generates X-rays upon reception of the electrons from the cathode between a first tube voltage and a second tube voltage higher than the first tube voltage. The grid control unit alternately switches the grid voltage applied between the cathode and a grid electrode that controls electrons emitted from the cathode to the anode between a first grid voltage and a second grid voltage higher than the first grid voltage. The grid control unit performs control to decrease a grid voltage to the first grid voltage and control to hold it exclusively within a first transition period in which the tube voltage makes a transition from the second tube voltage to the first tube voltage and a first hold period in which the tube voltage is held at the first tube voltage so as to avoid a tube current from exceeding the upper limit tube current based on allowable power.

The X-ray tube control system and the X-ray computed tomography imaging apparatus according to the present embodiment will be described in detail below with reference to the accompanying drawings.

The X-ray computed tomography imaging apparatus (CT apparatus) includes various types such as the third generation CT and the fourth generation CT. Any of these types can be applied to the present embodiment. In this case, the third generation CT is the Rotate/Rotate-Type designed to make an X-ray tube and a detector rotate around a subject. The fourth generation CT is the Stationary/Rotate-Type designed to make only an X-ray tube rotate about a subject while many X-ray detection elements arrayed in a ring shape are fixed.

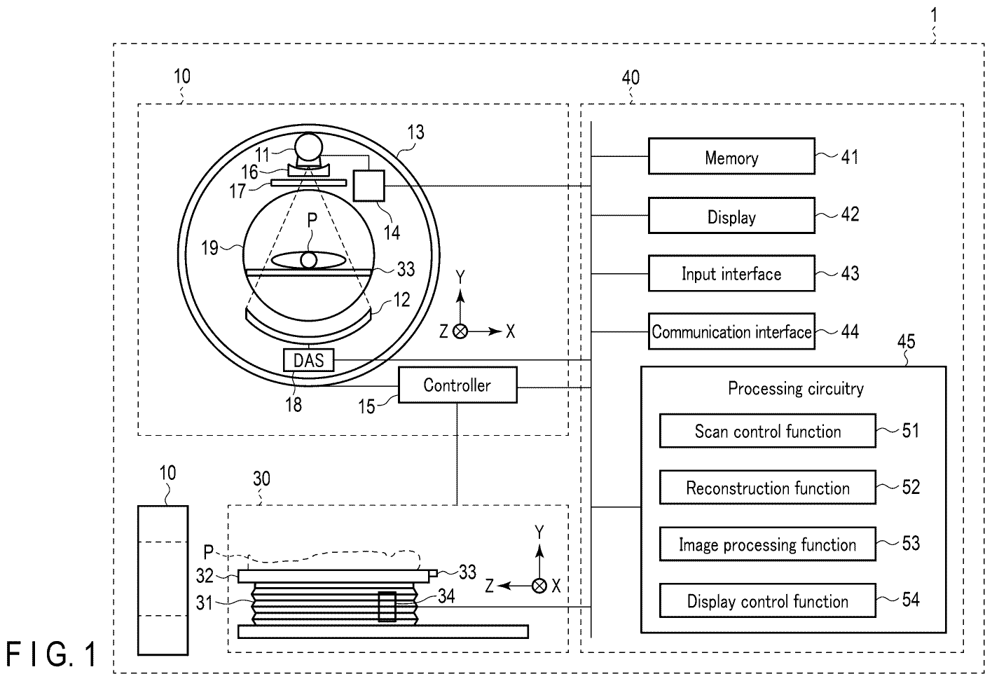

FIG. 1 is a view showing an example of the arrangement of an X-ray computed tomography imaging apparatus 1 according to the present embodiment. As shown in FIG. 1, the X-ray computed tomography imaging apparatus 1 includes a gantry 10, a bed 30, and a console 40. For the sake of descriptive convenience, FIG. 1 shows the plurality of gantries 10. In practice, however, one or a plurality of gantries may be used. The gantry 10 is a scan apparatus having an arrangement for performing X-ray CT imaging with respect to a subject P. The bed 30 is a transfer apparatus on which the subject P to be subjected to X-ray CT imaging is placed and which is configured to position the subject P. The console 40 is a computer that controls the gantry 10. For example, the gantry 10 and the bed 30 are installed in a CT examination room. The console 40 is installed in a control room adjacent to the CT examination room. The gantry 10, the bed 30, and the console 40 are communicably connected to each other wiredly or wirelessly. Note that the console 40 need not always be installed in a control room. For example, the console 40 may be installed in the same room as the gantry 10 and the bed 30. Alternatively, the console 40 may be incorporated in the gantry 10.

As shown in FIG. 1, the gantry 10 includes an X-ray tube I1, an X-ray detector 12, a rotating frame 13, an X-ray high voltage device 14, a controller 15, a wedge 16, a collimator 17, and a DAS (Data Acquisition System) 18.

The X-ray tube 11 irradiates the subject P with X-rays. More specifically, the X-ray tube 11 includes a cathode that generates thermal electrons, an anode that generates X-rays upon reception of the thermal electrons flying from the cathode, and a vacuum tube holding the cathode and the anode. The X-ray tube 11 is connected to the X-ray high voltage device 14 via a high voltage cable.

The X-ray high voltage device 14 applies a tube voltage between the cathode and the anode. Upon the application of the tube voltage, thermal electrons fly from the cathode to the anode. When the thermal electrons fly from the cathode to the anode, a tube current flows. When the thermal electrons collide with the anode, X-rays are generated.

The X-ray detector 12 detects the X-rays emitted from the X-ray tube 11 and transmitted through the subject P and outputs an electrical signal corresponding to the dose of detected X-rays to the data acquisition system 18. The X-ray detector 12 has a structure in which a plurality of X-ray detection element rows, each having a plurality of X-ray detection elements arrayed in the channel direction, are arrayed in the slice direction (column direction). The X-ray detector 12 is, for example, an indirect conversion type detector having a grid, a scintillator array, and an optical sensor array. The scintillator array has a plurality of scintillators. The scintillator outputs an amount of light corresponding to the dose of incident X-rays. The grid has an X-ray shielding plate arranged on the X-ray incident surface side of the scintillator array and configured to absorb scattered X-rays. Note that the grid is sometimes called a collimator (one-dimensional collimator or two-dimensional collimator). The optical sensor array converts light from each scintillator into an electrical signal corresponding to the amount of light. As an optical sensor, for example, a photodiode is used. Note that the X-ray detector 12 may be a direct conversion type detector.

The rotating frame 13 is an annular frame that supports the X-ray tube 11 and the X-ray detector 12 so as to allow them to rotate about the rotation axis (Z-axis). More specifically, the rotating frame 13 supports the X-ray tube 11 and the X-ray detector 12 so as to make them face each other. The rotating frame 13 is supported on a fixed frame (not shown) so as to be able to rotate about the rotation axis. The controller 15 rotates the rotating frame 13 about the rotation axis to rotate the X-ray tube 11 and the X-ray detector 12 about the rotation axis. The rotating frame 13 rotates about the rotation axis at a predetermined angular velocity upon reception of drive power from the drive mechanism of the controller 15. An FOV (Field Of View) is set in an opening portion 19 of the rotating frame 13.

In the present embodiment, the rotation axis of the rotating frame 13 in a non-tilt state or the longitudinal direction of a top plate 33 of the bed 30 is defined as the Z-axis direction, an axis direction that is orthogonal to the Z-axis direction and is horizontal to the floor surface is defined as the X-axis direction, and an axis direction that is orthogonal to the Z-axis direction and is vertical to the floor surface is defined as the Y-axis direction.

The X-ray high voltage device 14 includes a high voltage generator and an X-ray controller. The high voltage generator includes electrical circuitry such as a transformer and a rectifier and generates a high voltage applied to the X-ray tube 11 and a filament current supplied to the X-ray tube 11. The X-ray controller controls an output voltage corresponding to X-rays emitted by the X-ray tube 11. The high voltage generator may be based on a transformer scheme or inverter scheme. The X-ray high voltage device 14 may be provided on the rotating frame 13 in the gantry 10 or provided on a fixed frame (not shown) in the gantry 10.

The wedge 16 adjusts the dose of X-rays applied to the subject P. More specifically, the wedge 16 attenuates X-rays such that the dose of X-rays applied from the X-ray tube 11 to the subject P has a predetermined distribution. For example, as the wedge 16, a metal plate made of aluminum, such as a wedge filter or bow-tie filter, is used.

The collimator 17 limits the irradiation range of X-rays transmitted through the wedge 16. The collimator 17 slidably supports a plurality of lead plates that shield against X-rays and adjusts the form of the slit formed by the plurality of lead plates. Note that the collimator 17 is sometimes called an X-ray aperture.

The data acquisition system 18 reads out, from the X-ray detector 12, an electrical signal corresponding to the dose of X-rays detected by the X-ray detector 12. The data acquisition system 18 amplifies the read electrical signal and acquires detection data having a digital value corresponding to the dose of X-rays throughout a view period by integrating the electrical signal throughout the view period. Detection data is called projection data. The data acquisition system 18 is implemented by, for example, an ASIC (Application Specific Integrated Circuit) provided with a circuitry element that can generate projection data. Projection data is transmitted to the console 40 via a non-contact data transmitter or the like.

Although the present embodiment exemplifies the integral X-ray detector 12 and the X-ray computed tomography imaging apparatus 1 provided with the X-ray detector 12, the technique according to the present embodiment can also be applied to a photon counting X-ray detector.

The controller 15 controls the X-ray high voltage device 14 and the data acquisition system 18 to execute X-ray CT imaging in accordance with a scan control function 51 of a processing circuitry 45 of the console 40. The controller 15 includes a processing circuitry having a CPU (Central Processing Unit), MPU (Micro Processing Unit), or the like and a drive mechanism such as a motor and an actuator. The processing circuitry includes, as hardware resources, a processor such as a CPU and memories such as ROM (Read Only Memory) and a RAM (Random Access Memory).

The controller 15 executes various types of functions by using a processor that executes programs expanded in a memory. Note that the respective types of functions need not always be implemented by a single processing circuit. A processing circuitry may be formed by combining a plurality of independent processors, and each processor may implement a corresponding function by executing a corresponding program. The controller 15 may be implemented by an FPGA (Field Programmable Gate Array). Alternatively, the controller 15 may be implemented by another CPLD (Complex Programmable Logic Device) or SPLD (Simple Programmable Logic Device). The controller 15 has a function of controlling the operations of the gantry 10 and the bed 30 upon reception of input signals from an input interface 43 (to be described later) attached to the console 40 or the gantry 10. For example, the controller 15 performs control to rotate the rotating frame 13, control to tilt the gantry 10, and control to operate the bed 30 and the top plate 33 upon reception of input signals. Note that the controller 15 implements control to tilt the gantry 10 by rotating the rotating frame 13 about an axis parallel to the X-axis direction in accordance with tilt angle information input by the input interface attached to the gantry 10. Note that the controller 15 may be provided on the gantry 10 or the console 40.

The bed 30 includes a base 31, a support frame 32, the top plate 33, and a bed drive device 34. The base 31 is installed on the floor surface. The base 31 is a housing that supports the support frame 32 so as to allow it to move in the vertical direction (Y-axis direction) with respect to the floor surface. The support frame 32 is a frame provided on the upper portion of the base 31. The support frame 32 supports the top plate 33 so as to allow it to slide along the rotation axis (Z-axis). The top plate 33 is a flexible plate on which the subject P is placed. The bed drive device 34 is accommodated in the housing of the bed 30. The bed drive device 34 is a motor or actuator that generates drive power for moving the support frame 32 and the top plate 33 on which the subject P is placed. The bed drive device 34 operates under the control of the console 40 and the like.

The console 40 includes a memory 41, a display 42, the input interface 43, a communication interface 44, and the processing circuitry 45. Data communication is performed among the memory 41, the display 42, the input interface 43, the communication interface 44, and the processing circuitry 45 via a bus (BUS). Although the console 40 will be described as being separate from the gantry 10, the gantry 10 may include the console 40 or part of the constituent elements of the console 40.

The memory 41 is a storage device such as an HDD (Hard Disk Drive), an SSD (Solid State Drive), or an integrated circuit storage device, which stores various types of information. The memory 41 may be a portable storage medium such as a CD (Compact Disc), a DVD (Digital Versatile Disc), a BD (Blue-ray® Disc), a flash memory, or the like. The memory 41 may be a drive device that reads and writes various types of information between semiconductor memory elements such as a flash memory and a RAM. In addition, the save area of the memory 41 may be located in the X-ray computed tomography imaging apparatus 1 or in an external storage device connected via a network. The memory 41 stores, for example, projection data and reconstruction image data.

The display 42 displays various types of information. For example, the display 42 outputs the CT image generated by the processing circuitry 45, a GUI (Graphical User Interface) for accepting various types of operations from the operator, and the like. As the display 42, various types of arbitrary displays can be used as needed. For example, as the display 42, an LCD (Liquid Crystal Display), a CRT (Cathode Ray Tube), an OELD (Organic Electro Luminescence Display), or a plasma display can be used.

Note that the display 42 may be provided in any place in the control room. The display 42 may be provided on the gantry 10. The display 42 may be of a desktop type or may be composed of a tablet terminal or the like wirelessly communicable with the main body of the console 40. As the display 42, one or two or more projectors may be used.

The input interface 43 accepts various types of input operations from the operator, converts the accepted input operations into electrical signals, and outputs them to the processing circuitry 45. For example, the input interface 43 accepts acquisition conditions for the acquisition of projection data, reconstruction conditions for the reconstruction of a CT image, image processing conditions for the generation of a postprocessing image from the CT image, and the like from the operator. As the input interface 43, for example, a mouse, a keyboard, a trackball, switches, buttons, a joystick, a touch pad, a touch panel display, and the like can be used as needed. Note that in the present embodiment, the input interface 43 is not limited to one that includes physical operation components such as a mouse, a keyboard, a trackball, switches, buttons, a joystick, a touch pad, and a touch panel display. An example of the input interface 43 includes an electrical signal processing circuit that receives an electrical signal corresponding to an input operation from an external input device provided separately from the apparatus and outputs the electrical signal to the processing circuitry 45. The input interface 43 may be provided on the gantry 10. The input interface 43 may be composed of a tablet terminal or the like which can wirelessly communicate with the main body of the console 40.

The communication interface 44 includes a NIC (Network Interface Card) for communicating various types of data with an external device such as a workstation, a PACS (Picture Archiving and Communication Systems), a RIS (Radiology Information System), or a HIS (Hospital Information System) via a network.

The processing circuitry 45 controls the overall operation of the X-ray computed tomography imaging apparatus 1 in accordance with an electrical signal corresponding to an input operation which is output from the input interface 43. The processing circuitry 45 generates image data based on the electrical signal output from the X-ray detector 12. For example, the processing circuitry 45 includes a processor such as a CPU, MPU, or GPU and memories such as a ROM and a RAM as hardware resources. The processing circuitry 45 executes the scan control function 51, a reconstruction function 52, an image processing function 53, a display control function 54, and the like by using a processor that executes programs expanded in the memory.

Note that the functions 51 to 54 need not always be implemented by a single processing circuit. A processing circuitry may be formed by combining a plurality of independent processors, and each processor may implement a corresponding one of the functions 51 to 54 by executing a corresponding program.

With the scan control function 51, the processing circuitry 45 executes dual energy scan by controlling the X-ray high voltage device 14, the controller 15, and the data acquisition system 18 in accordance with preset scan conditions. The scan conditions to be set include a tube voltage, a grid voltage, information indicating whether to perform tube current modulation, the rotational speed of the rotating frame 13, a scan range, a scan region, and the like.

With the reconstruction function 52, the processing circuitry 45 performs preprocessing such as logarithmic conversion processing, offset correction processing, sensitivity correction processing between channels, beam hardening correction, and interpolation processing for data missing due to tube voltage switching with respect to the projection data output from the data acquisition system 18. The processing circuitry 45 generates a CT image (to be referred to as a reference material image hereinafter) by performing material discrimination for the preprocessed projection data and performing reconstruction processing for the projection data after the material discrimination. As reconstruction processing, reconstruction processing to which a filter correction back projection method, a successive approximation reconstruction method, and machine learning are applied can be used. Note that the processing circuitry 45 may generate a CT image (to be also referred to as an integral image) by performing reconstruction processing for projection data without material discrimination.

With the image processing function 53, the processing circuitry 45 converts the CT image generated by the reconstruction function 52 into a section image of an arbitrary section or a rendering image in an arbitrary viewpoint direction. The conversion is performed based on an input operation accepted from the operator via the input interface 43. For example, the processing circuitry 45 generates a rendering image in an arbitrary viewpoint direction by performing three-dimensional image processing such as volume rendering, surface volume rendering, image value projection processing, MPR (Multi-Planer Reconstruction) processing, or CPR (Curved MPR) with respect to the CT image. Note that the reconstruction function 52 may directly generate a rendering image in an arbitrary viewpoint direction.

With the display control function 54, the processing circuitry 45 displays various types of information on the display 42. For example, the processing circuitry 45 displays various types of images generated by the image processing function 53 on the display 42.

Although the console 40 is described as a single console that executes a plurality of functions, different consoles may respectively execute a plurality of functions. The processing circuitry 45 need not always be included in the console 40 and may be included in a comprehensive server that comprehensively performs processing for the projection data acquired by a plurality of medical image diagnosis apparatuses. Postprocessing may be performed by either the console 40 or an external workstation. In addition, the console 40 and the workstation may concurrently perform postprocessing.

An X-ray tube control system 100 including the X-ray tube 11 and the X-ray high voltage device 14 according to the present embodiment will be described. Assume that the X-ray tube control system 100 is mounted on the X-ray computed tomography imaging apparatus 1.

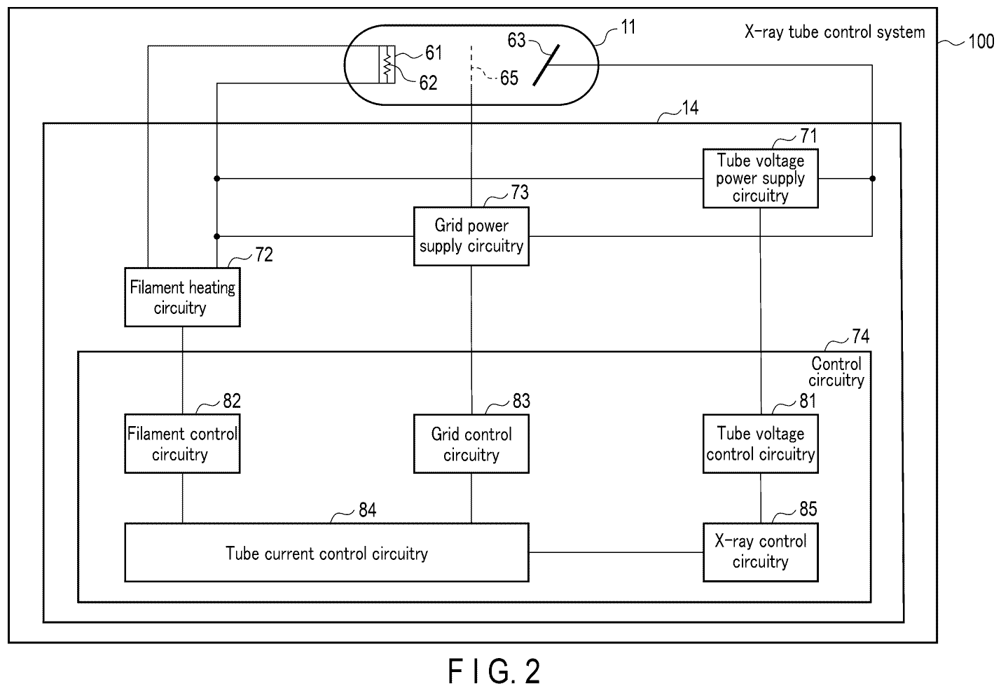

FIG. 2 is a block diagram showing an example of the arrangement of the X-ray tube control system 100 according to the present embodiment. As shown in FIG. 2, the X-ray tube control system 100 includes the X-ray tube 11 and the X-ray high voltage device 14.

The X-ray tube 11 is a vacuum vessel accommodating a cathode 61, an anode 63, and a grid electrode 65. The cathode 61 emits electrons. More specifically, the cathode 61 has a filament 62 formed of, for example, a metal such as tungsten or nickel having a fine linear shape. The cathode 61 is connected to the X-ray high voltage device 14 via a cable or the like. The filament 62 generates heat and emits electrons (thermal electrons) upon reception of a current (to be referred to as a filament current hereinafter) for heating from the X-ray high voltage device 14.

The anode 63 is an electrode formed of a heavy metal such as tungsten or molybdenum and having a disk shape. The anode 63 rotates accompanying the rotation of the rotor (not shown) about the axis. The X-ray high voltage device 14 applies a high tube voltage between the cathode 61 and the anode 63. The electrons emitted from the cathode 61 fly and collide with the anode 63 owing to the effect of the tube voltage. When electrons flow from the cathode 61 to the anode 63, a tube current flows. The anode 63 generates X-rays upon reception of the electrons. The range on the anode 63 in which electrons collide with the anode 63 forms a focus.

The grid electrode 65 is an electrode placed between the cathode 61 and the anode 63. The grid electrode 65 controls electrons propagating from the cathode 61 to the anode 63. More specifically, the X-ray high voltage device 14 applies a grid voltage corresponding to the cathode potential to the grid electrode 65.

As shown in FIG. 2, the X-ray high voltage device 14 includes a tube voltage power supply circuitry 71, a filament heating circuitry 72, a grid power supply circuitry 73, and a control circuitry 74. The tube voltage power supply circuitry 71, the filament heating circuitry 72, the grid power supply circuitry 73 each are connected to the control circuitry 74 via a signal line and the like.

The tube voltage power supply circuitry 71 generates a tube voltage applied between the cathode 61 and the anode 63 under the control of the control circuitry 74. For example, in the case of an inverter type X-ray high voltage device, the tube voltage power supply circuitry 71 includes an AC/DC converter that converts an AC voltage from a commercial power supply into a DC voltage, an inverter that converts the DC voltage from the AC/DC converter into an AC voltage, a transformer that steps up the AC voltage from the inverter, and a high voltage rectifying/smoothing circuitry that generates a high DC voltage by rectifying and smoothing the AC voltage stepped up by the transformer. The high DC voltage from the high voltage rectifying/smoothing circuitry is applied as a tube voltage between the cathode 61 and the anode 63.

The filament heating circuitry 72 supplies a filament current to the filament 62 under the control of the control circuitry 74. When the filament current is supplied to the filament 62, the filament 62 is heated to emit the number of electrons corresponding to the temperature of the filament 62. Accordingly, controlling the filament current can control a tube current. The filament heating circuitry 72 may be implemented by a step-down circuitry that steps down the voltage generated by the tube voltage power supply circuitry 71 or may be implemented by a power supply system independent of the tube voltage power supply circuitry 71.

The grid power supply circuitry 73 applies a grid voltage between the cathode 61 and a grid electrode 135 under the control of the control circuitry 74. Applying the grid voltage will adjust the amount of electrons from the filament 62 to the anode 63. Accordingly, controlling the grid voltage can control the tube current. The grid power supply circuitry 73 may be implemented by a step-down circuitry that steps down the voltage generated by the tube voltage power supply circuitry 71 or a power supply system independent of the tube voltage power supply circuitry 71.

The control circuitry 74 is a processor that controls the tube voltage power supply circuitry 71, the filament heating circuitry 72, and the grid power supply circuitry 73. More specifically, the control circuitry 74 includes a tube voltage control circuitry 81, a filament control circuitry 82, a grid control circuitry 83, a tube current control circuitry 84, and an X-ray control circuitry 85.

The tube voltage control circuitry 81 controls the tube voltage applied between the cathode 61 and the anode 63 under the control of the X-ray control circuitry 85. More specifically, the tube voltage control circuitry 81 alternately switches the tube voltage between the first tube voltage and the second tube voltage higher than the first tube voltage. The first tube voltage will be referred to as a low tube voltage or simply a tube voltage hereinafter. The second tube voltage will be referred to as a high tube voltage or simply a high voltage hereinafter. Alternate switching between the low tube voltage and the high tube voltage is also called kV switching.

More specifically, the tube voltage control circuitry 81 executes feedback control for the tube voltage by controlling the tube voltage power supply circuitry 71 based on the difference between an actually measured tube voltage and a set tube voltage. The actually measured tube voltage is detected by a tube voltage detector (not shown). The tube voltage detector detects the voltage applied between the cathode 61 and the anode 63 as an actually measured tube voltage. The crest value data of the detected actually measured voltage is supplied to the control circuitry 74. The tube voltage detector is connected between the tube voltage power supply circuitry 71 and the anode 63. The set tube voltage means the target value of the tube voltage designated by the user or the like or determined by the processing circuitry 45. The data of a set tube voltage is supplied from the X-ray control circuitry 85. In dual energy scan, the set tube voltage of the low tube voltage (to be referred as the set low tube voltage hereinafter) and the set tube voltage of the high tube voltage (to be referred to as the set high tube voltage hereinafter) are designated. The tube voltage control circuitry 81 instructs the tube voltage power supply circuitry 71 to step down or step up the tube voltage so as to null the difference between the actually measured tube voltage and the set low tube voltage at the time of applying the low tube voltage and so as to null the difference between the actually measured tube voltage and the set high tube voltage at the time of applying the high tube voltage. The tube voltage power supply circuitry 71 adjusts the tube voltage in accordance with an instruction from the tube voltage control circuitry 81. With this operation, feedback control of the tube voltage is performed.

The filament control circuitry 82 controls a filament current that heats the filament 62 of the cathode 61. Filament current control schemes include a control scheme of supplying a constant filament current (to be referred to as a constant If scheme hereinafter) and a tube current feedback control scheme of controlling the tube current to a desired value. In the case of the constant If scheme, the filament control circuitry 82 instructs the filament heating circuitry 72 concerning the supply amount of filament current so as to supply a set filament current to the filament 62. The set filament current means the set value of the filament current necessary to supply a target tube current. A set filament current is designated by the user or the like or determined by the processing circuitry 45. The filament heating circuitry 72 supplies a filament current to the filament 62 in accordance with an instruction from the filament control circuitry 82.

In the case of the tube current feedback control scheme, the filament control circuitry 82 executes tube current feedback control by controlling the filament heating circuitry 72 based on the difference between an actually measured tube current and a target tube current. An actually measured tube current is detected by a tube current detector (not shown). The tube current detector detects, as a tube current, a current flowing due to the flowing of thermal electrons from the cathode 61 to the anode 63. The data of the crest value of the detected actually measured tube current is supplied to the control circuitry 74. The tube current detector is connected between the filament heating circuitry 72 and the filament 62. The target tube current means the set value of the tube current designated by the user or the like or determined by the processing circuitry 45. The data of the target tube current is supplied from the X-ray control circuitry 85. The filament control circuitry 82 instructs the filament heating circuitry 72 to decrease or increase a filament current so as to null the difference between the actually measured tube current and the target tube current. The filament heating circuitry 72 adjusts the filament current in accordance with an instruction from the filament control circuitry 82. With this operation, tube current feedback control is performed.

The grid control circuitry 83 controls the grid voltage applied between the cathode 61 and the grid electrode 65. For example, the grid control circuitry 83 alternately switches the grid voltage between the first grid voltage and the second grid voltage. The first grid voltage will be referred to as a low grid voltage or simply a low voltage hereinafter. The second grid voltage will be referred to as a high grid voltage or simply a high voltage hereinafter. Note that the grid voltage need not always be set in two steps and may be set in three or more steps. The grid control circuitry 83 controls the grid power supply circuitry 73 in synchronism with switching between the low tube voltage and the high tube voltage by the tube voltage control circuitry 81 so as to apply the low grid voltage at the time of applying the low tube voltage and so as to apply the high grid voltage at the time of applying the high tube voltage. More specifically, the grid control circuitry 83 performs control to decrease the grid voltage to the low grid voltage and control to hold exclusively within the first transition period in which the tube voltage makes a transition from the high tube voltage to the low tube voltage and a first hold period in which the tube voltage is held at the low tube voltage so as to avoid a tube current from exceeding the upper limit tube current based on allowable power. From another point of view, the grid control circuitry 83 performs control to hold the grid voltage at the low grid voltage exclusively within the first hold period. In detail, “low grid voltage” means a negative grid voltage with a low absolute value, and “high grid voltage” means a negative grid voltage with a high absolute value.

More specifically, the grid power supply circuitry 73 executes tube current feedback control by controlling the grid power supply circuitry 73 based on the difference between an actually measured tube voltage and a target tube current. As a result of the tube current feedback control, it is possible to alternately switch the grid voltage between the low voltage and the high voltage. In the case of dual energy scan, as target tube currents, the first tube current and the second tube current larger than the first tube current are set. The first tube current will be referred to as a low tube current or simply a low current hereinafter. The second tube current will be referred as a high tube current or simply a high current hereinafter. The target tube current of the low tube current will be referred to as a target low tube current. The target tube current of the high tube current will be referred as a target high tube current. The grid control circuitry 83 instructs the grid power supply circuitry 73 to drop or step down or step up the grid voltage so as to null the difference between the actually measured tube voltage and the target high tube current at the time of applying the low tube voltage or so as to null the difference between the actually measured tube current and the target low tube current at the time of applying the high tube voltage. The grid power supply circuitry 73 adjusts the grid voltage in accordance with an instruction from the grid control circuitry 83. The grid voltage can be alternately switched between the high tube voltage and the low tube voltage.

The tube current control circuitry 84 controls the filament control circuitry 82 and the grid control circuitry 83 under the control of the X-ray control circuitry 85. For example, the tube current control circuitry 84 can switch between the filament current control scheme and the constant If scheme by using the filament control circuitry 82 in accordance with an instruction from the X-ray control circuitry 85. Another example is that the tube current control circuitry 84 can switch between tube current control by the filament control circuitry 82 and tube current control by the grid control circuitry 83 at the time of applying the high tube voltage and applying the low tube voltage.

The X-ray control circuitry 85 functions as the main unit of the X-ray tube control system 100. For example, the X-ray control circuitry 85 notifies the tube voltage control circuitry 81 and the tube current control circuitry 84 of various types of scan conditions, the kV switching scheme, and various types of signals from outside of the X-ray tube control system 100.

An operation example of the X-ray tube control system 100 will be described below.

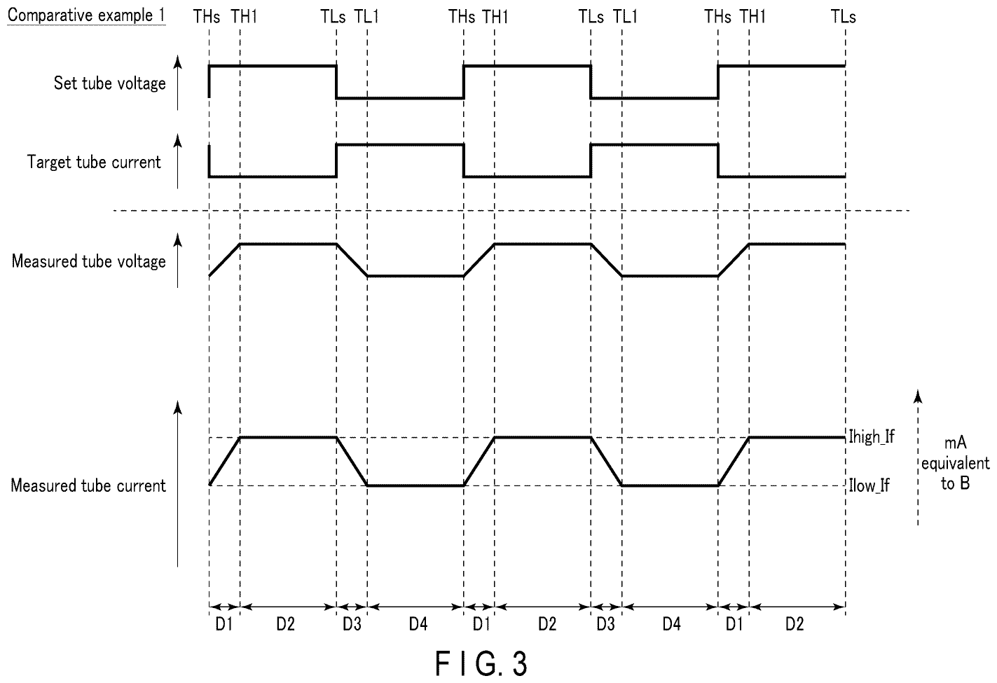

First of all, problems in kV switching in Comparative Example 1 will be described in detail with reference to FIG. 3. FIG. 3 is a timing chart of kV switching in Comparative Example 1. Comparative Example 1 assumes that the X-ray tube 11 is not equipped with the grid electrode 65. The vertical axis in FIG. 3 and other timing charts represents absolute values.

As indicated by the upper plots in FIG. 3, the set tube voltage is alternately switched instantaneously between the high tube voltage and the low tube voltage every time the rotating frame 13 rotates through a predetermined angle. More specifically, the tube voltage control circuitry 81 receives, from the controller 15, a trigger signal generated every time the rotating frame 13 rotates through a fine angle and alternately supplies an instruction to increase the tube voltage and an instruction to decrease the tube voltage to the tube voltage power supply circuitry 71, every time a predetermined number of trigger signals are received. The tube voltage power supply circuitry 71 instantaneously switches the set tube voltage to the high tube voltage at time THs when an increase instruction is issued and instantaneously switches the set tube voltage to the low tube voltage at time TLs when a decrease instruction is issued.

The target tube current according to Comparative Example 1 means an ideal tube current. As indicated by the target tube current, it is ideal that the tube current is alternately switched simultaneously between the low tube current and the high tube current in synchronism with tube voltage switching to make the low tube current flow at the time of applying the high tube voltage and to make the high tube current flow at the time of applying the low tube voltage so as to uniform the X-ray dose at the time of applying the high tube voltage and at the time of applying the low tube voltage.

The lower plots in FIG. 3 schematically represent the transition between the actually measured tube voltage and the actually measured current. As indicated by the lower plots in FIG. 3, the actually measured tube voltage follows the set tube voltage with a delay. More specifically, the actually measured tube voltage starts to increase from time THs toward the high tube voltage and reaches the high tube voltage at time TH1. In addition, the actually measured tube voltage starts to decrease from time TLs to the low tube voltage and reaches the low tube voltage at time TL1. Likewise, the actually measured tube voltage is alternately switched between the high tube voltage and the low tube voltage. Assume that a period (second transition period) in which the tube voltage makes a transition from the low voltage to the high voltage, in other words, a period from time THs to time TH1, will be referred to as an increase period D1, a period (second hold period) in which the tube voltage is held at the high voltage, in other words, a period from time TH1 to time TLs, will be referred to as a high kV period D2, a period (first transition period) in which the tube voltage makes a transition from the high voltage to the low voltage, in other words, a period from time TLs to time TL1, will be referred to as a decrease period D3, and a period (first hold period) in which the tube voltage is held at the low voltage, in other words, a period from time TL1 to time THs, will be referred to as a low kV period D4.

Referring to the waveform graph of the actually measured tube current, the left ordinate represents the actually measured tube current [mA], and the right ordinate represents If equivalent mA. “If equivalent mA” means a tube current flowing according to emission characteristics based on a given tube voltage or a given filament temperature. Ihigh_If represents a tube current corresponding to a filament temperature at the time of applying the high tube voltage, and Ilow_If represents a tube current corresponding to a filament temperature at the time of applying the low tube voltage.

If there is no grid electrode and no feedback control for a filament current is performed as in Comparative Example 1, the actually measured tube current varies in accordance with the filament temperature, and the acceleration of thermal electrons varies in accordance with the actually measured tube voltage. Accordingly, as the actually measured tube voltage drops, the actually measured tube current also decreases. This makes it impossible to hold the uniformity of the X-ray dose in the high kV period D2 and the low kV period D4, resulting in difficulty in holding the uniformity of the quality of the projection data acquired in the high kV period D2 and the projection data acquired in the low kV period D4.

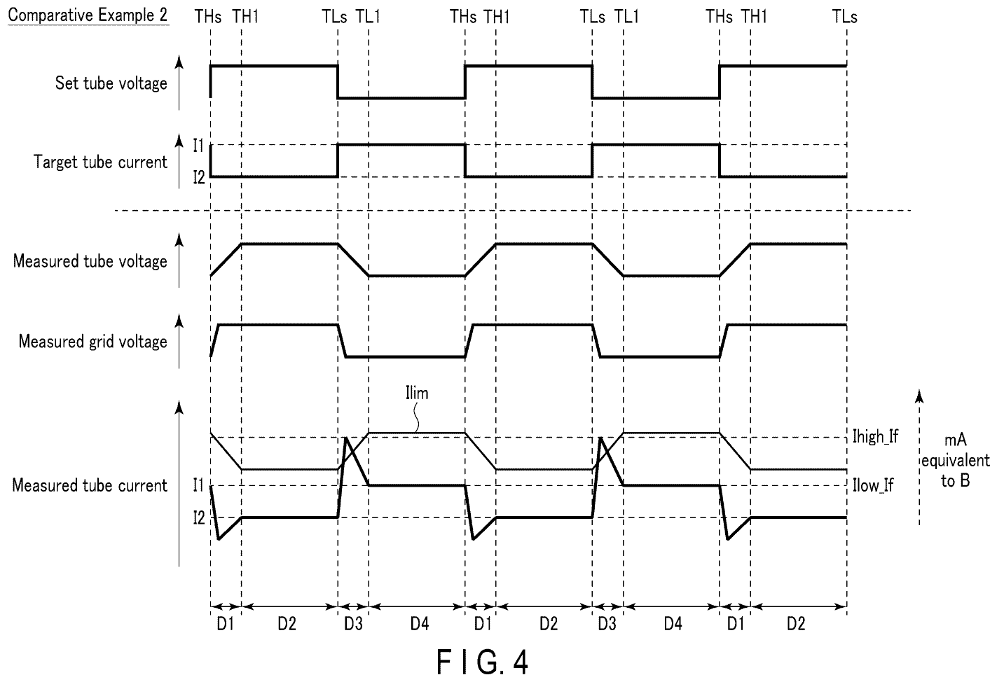

Problems in kV switching in Comparative Example 2 will be described in detail next with reference to FIG. 4. FIG. 4 is a timing chart of kV switching in Comparative Example 2. The lower plots in FIG. 4 schematically represent the transitions of the actually measured tube voltage, the actually measured grid voltage, and the actually measured tube current. In Comparative Example 2, the X-ray tube 11 is equipped with the grid electrode 65. The grid control circuitry 83 according to Comparative Example 2 switches the set grid voltage at the same timing as the switching of the set tube voltage. The behaviors of the set tube voltage, the target tube current, and the actually measured tube voltage in Comparative Example 2 are similar to those in Comparative Example 1. Note that the target tube current makes alternate transitions between a high tube current I1 and a low tube current I2.

As indicated by the lower plots in FIG. 4, the grid control circuitry 83 switches the grid voltage in synchronism with the switching of the target tube current. More specifically, the tube current control circuitry 84 supplies an instruction to increase the tube current to the grid control circuitry 83 at time TLs (the time when the target tube voltage is switched from the high voltage to the low voltage) when the target tube current is switched from the low current I2 to the high current I1. Upon reception of the instruction, the grid control circuitry 83 sets the target tube current to the target high tube current I1 and applies the grid voltage that sets the tube current to the high tube current I1 at the time of the low voltage. As a result, the actually measured grid voltage starts to drop. Accordingly, the actually measured tube current starts to increase. After TL1, the actually measured grid voltage is held at the low voltage so as to maintain the actually measured tube current at the high tube current I1.

In contrast, the tube current control circuitry 84 supplies, to the grid control circuitry 83, an instruction to decrease the tube current at time THs (a time when the target tube voltage is switched from the low voltage to the high voltage) when the target tube current is switched from the high current to the low current. Upon reception of the instruction, the grid control circuitry 83 sets the target tube current to the low tube current I2 and applies a grid voltage that becomes the low tube current I2 at the time of the high voltage. As a result, the actually measured grid voltage starts to increase. Accordingly, the actually measured tube current starts to decrease. From TH1, the actually measured grid voltage is held at the high voltage so as to maintain the actually measured tube current at the low tube current I2.

The target low tube current I2 and the target high tube current I1 each are preferably determined to be a value that sets the same X-ray dose in the high kV period D2 and the low kV period D4. The high grid voltage corresponds to a grid voltage required to make the target low tube current I2 flow under the high tube voltage. The low grid voltage corresponds to a grid voltage required to make the target high tube current I1 flow under the low tube voltage. Note that the target high tube current I1 is equal to a tube current Ilow_If.

As shown in FIG. 4, since the tube voltage is a few orders of magnitude larger than the grid voltage, the increase period D1 and the decrease period D3 of the actually measured tube voltage tend to longer than the increase period and the decrease period of the actually measured grid voltage. If, for example, the tube voltage and the grid voltage simultaneously start to drop, the grid voltage decreases to the low grid voltage in the decrease period D3 in which the actually measured tube voltage is not held at the low tube voltage. As a result, an actually measured tube current larger than the high tube current I1 may temporarily flow. Likewise, if the tube voltage and the grid voltage simultaneously start to increase, an actually measured tube current lower than the low tube current I2 flow in the increase period D1.

If the actually measured tube current jumps to exceed an upper limit tube current Ilim in the decrease period D3, the allowable power of the X-ray tube 11 is exceeded, and various types of devices of the X-ray tube control system 100 may be damaged. The upper limit tube current Ilim means the tube current determined based on the actually measured tube voltage and the allowable power. The upper limit tube current Ilim can be obtained by, for example, dividing the allowable power by the actually measured tube voltage. The allowable power is the upper limit power within which the X-ray tube 11 and the like are not damaged and is determined by the focus size, the output of the X-ray high voltage device 14, the power supply power of the tube voltage power supply circuitry 71, and the like. The upper limit tube current Ilim shown in FIG. 4 schematically represents the upper limit tube current obtained by dividing the allowable power by the actually measured tube voltage. The upper limit tube current Ilim exhibits an opposite phase relationship with the actually measured tube voltage.

The present embodiment provides the X-ray tube control system 100 that can hold the X-ray dose uniformity in the high kV period D2 and the low kV period D4 while avoiding an actually measured tube current exceeding the upper limit tube current from flowing in the decrease period D3. The present embodiment will be described separately in Examples 1 to 5. Example 1 as a basic form of kV switching according to the present embodiment will be described first.

### Example 1

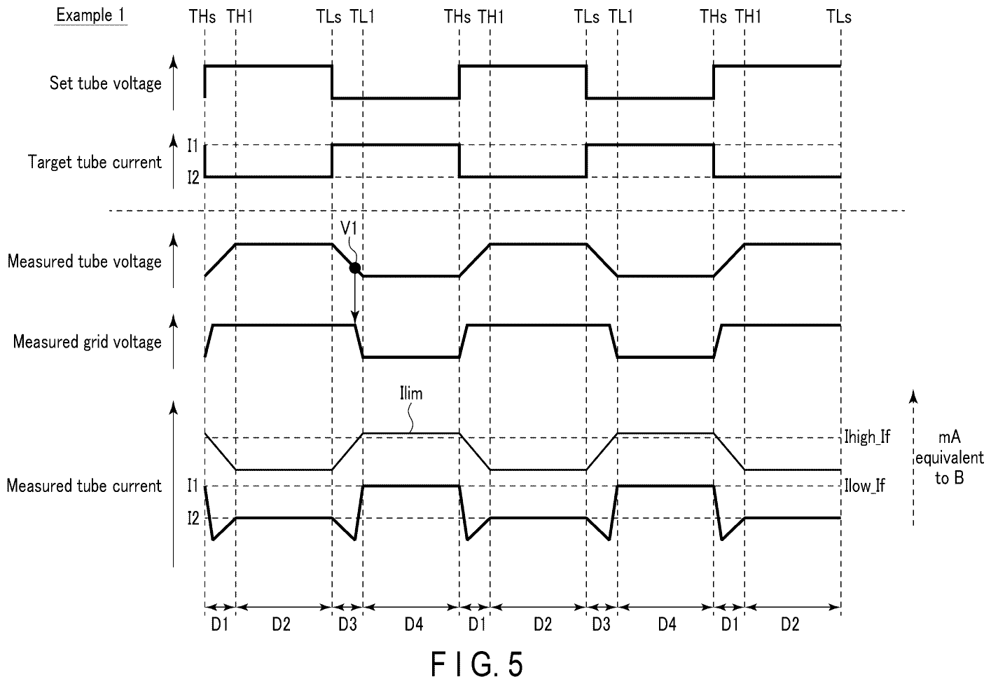

FIG. 5 is a timing chart of kV switching in Example 1. As shown in FIG. 5, the grid control circuitry 83 performs control to decrease the actually measured grid voltage to the low grid voltage and control to hold it exclusively within the decrease period D3 and the low kV period D4 so as to avoid the actually measured tube current from exceeding the upper limit tube current Ilim in the low kV period D4 in which the tube voltage is held at the low tube voltage. The meaning “performs control to decrease the actually measured grid voltage to the low grid voltage and control to hold it exclusively within the decrease period D3 and the low kV period D4” is that the actually measured grid voltage is not held at the low grid voltage in the decrease period D3 but is held at the low grid voltage for the first time in the low kV period D4 unlike in Comparative Example 2 in which the actually measured grid voltage reaches the low grid voltage in the decrease period D3 immediately before the low kV period D4, and the low grid voltage is held until the low kV period D4. As described later, the start of the decreasing of the actually measured grid voltage is not limited to the decrease period D3 and may be in the low kV period D4. In this case, both control to decrease the actually measured grid voltage to the low grid voltage and control to hold it are performed exclusively within the low kV period D4. In other words, the grid control circuitry 83 performs control to hold the actually measured grid voltage at the low grid voltage exclusively within the low kV period D4.

The decreasing of the grid voltage will be described in detail. The grid control circuitry 83 performs tube current feedback control by adjusting the grid voltage. The grid control circuitry 83 according to Example 1 does not immediately execute tube current feedback control based on the difference between the high tube current I1 and the actually measured tube current even if the tube current control circuitry 84 supplies an instruction to increase the tube current at time TLs (the time when the target tube voltage is switched from the high voltage to the low voltage) when the target tube current is switched from the low tube current I2 to the high tube current I1. Even if an instruction to increase the tube current is supplied, the grid control circuitry 83 maintains the high grid voltage that is the actually measured grid voltage at time TLs immediately before the supply of the instruction.

Accordingly, the actually measured tube current is lower than the low tube current I2 except for an end part of the decrease period D3. The start time point of the decreasing of the actually measured grid voltage according to Example 1 is temporally shifted backward with respect to time TLs up to time TL1 when the target tube voltage reaches the low voltage.

For example, the grid control circuitry 83 starts to decrease the grid voltage in response to a decrease in actually measured tube voltage from the high voltage to a reference value V1 after the lapse of time TLs. More specifically, when the tube voltage control circuitry 81, which is monitoring an actually measured tube voltage, detects a decrease in actually measured tube voltage to the reference value V1, the tube voltage control circuitry 81 supplies a corresponding signal to the grid control circuitry 83 via the X-ray control circuitry 85 and the tube current control circuitry 84. Upon reception of the signal, the grid control circuitry 83 sets the target tube current to the high tube current I1 and controls the grid voltage in accordance with tube current feedback based on the difference between the high tube current I1 and the actually measured tube current. As a result, the actually measured grid voltage starts to drop from the time when it reaches the reference value V1. When the actually measured grid voltage reaches the low voltage, the low voltage is held. Holding the actually measured grid voltage at the low voltage will hold the actually measured tube current at the high tube current I1 or the tube current Ilow_If.

The reference value V1 means a tube voltage value that makes the grid voltage drop and is set to a value between the high tube voltage and the low tube voltage. More specifically, the reference value V1 can be calculated by measuring in advance a period in which the actually measured grid voltage drops from the high grid voltage to the low grid voltage and adding an estimated tube voltage amount by which the tube voltage can drop in the measured period to the low tube voltage. Note that the grid control circuitry 83 may start to decrease the grid voltage in response to a decrease in actually measured tube voltage from the high tube voltage to the low tube voltage. In this case, both control to decrease the actually measured grid voltage to the low grid voltage and control to hold it are performed exclusively within the low kV period D4.

According to Example 1, the actually measured grid voltage is decreased to the low grid voltage and held at the low grid voltage exclusively within the low kV period D4. This makes it possible to prevent the actually measured tube current from exceeding the upper limit tube current Ilim or reduce the actually measured tube current in the decrease period D3 and hence to prevent or reduce damage to devices mounted on the X-ray tube control system 100. This can make the low tube current flow at application of the high tube voltage and make the high tube current flow at the time of application of the low tube voltage, thereby reducing the difference between the X-ray dose at the time of application of the high tube voltage and the X-ray dose at the time of application of the low tube voltage. This eventually makes it possible to improve the uniformity of the quality of the projection data acquired at the application of the high tube voltage and the projection data acquired at the application of the low tube voltage.

The grid voltage is increased in a manner similar to Comparative Example 2. Even if an instruction to decrease the tube current is supplied, the grid control circuitry 83 maintains the low grid voltage that is the actually measured grid voltage at time THs immediately before the supply of the instruction. Accordingly, the actually measured tube current is lower than the low tube current I2 except for an end part of the period D1. That is, the grid control circuitry 83 changes the grid voltage to a grid voltage that supplies the high tube voltage and the low tube current immediately at time THs when an instruction to decrease the tube current is issued. As a result, the actually measured grid voltage starts to increase from time THs. When the grid voltage reaches the high voltage, the high voltage is held. Holding the actually measured grid voltage at the high voltage will hold the actually measured tube current at the low tube current I2.

### Example 2

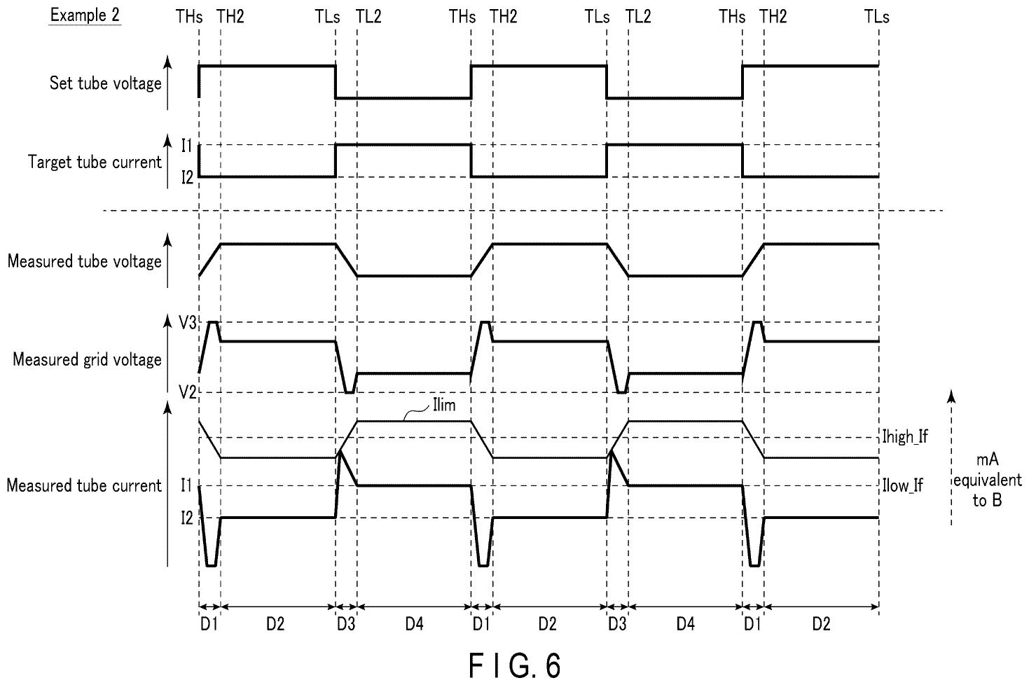

FIG. 6 is a timing chart of kV switching in Example 2. Example 2 is a developed modification of Example 1. As shown in FIG. 6, the grid control circuitry 83 decreases the actually measured grid voltage so as to make the tube current equal to or more than the high tube current I1 flow up to the upper limit tube current Ilim in the decrease period D3 in which the tube voltage makes a transition from the high tube voltage to the low tube voltage. The upper limit tube current Ilim is defined to the upper limit value of a tube current that does not exceed the allowable power under the current actually measured tube voltage. More specifically, the upper limit tube current Ilim is set to the value obtained by dividing the allowable power by the actually measured tube voltage. Note that in Example 2, the grid control circuitry 83 performs control to decrease the actually measured grid voltage to the low grid voltage and control to hold it exclusively within the decrease period D3 and the low kV period D4 to prevent the actually measured tube current from exceeding the upper limit tube current Ilim. The grid control circuitry 83 performs control to hold the actually measured grid voltage at the low grid voltage exclusively within the low kV period D4. Note that “control to decrease the grid voltage to the low grid voltage” according to Example 2 includes control to increase the grid voltage from the voltage V2 to the low grid voltage after performing control to actively decrease the actually measured grid voltage from the high grid voltage to the voltage V2 lower than the low grid voltage unlike in Example 1 of performing control to decrease the actually measured grid voltage from the high grid voltage to the low grid voltage.

The dropping of the actually measured grid voltage will be described in detail. The grid control circuitry 83 sets the target tube current to the upper limit tube current Ilim or a smaller value immediately at time TLs when an instruction to increase the tube current is issued and controls the grid voltage in accordance with tube current feedback based on the difference between the upper limit tube current Ilim or a smaller value and the actually measured tube current. In the decrease period D3, the grid control circuitry 83 decreases the actually measured grid voltage to the voltage V2 lower than the low grid voltage and then increases the low grid voltage by the low kV period D4. The actually measured grid voltage can be decreased to the voltage V2 by, for example, amplifying the difference between the upper limit tube current or a smaller value and the actually measured tube current. With the change in the grid voltage, the actually measured tube current increases from the low tube current to the upper limit tube current Ilim in the decrease period D3 and then decreases to the high tube current I1 in the low kV period D4. Rapidly decreasing the grid voltage to the voltage V2 in the decrease period D3 makes it possible to increase the tube current to a current higher than the high tube current I1 up to the upper limit tube current Ilim.

Increasing the tube current will promote the discharging of electric charge accumulated in the tube voltage power supply circuitry 71 and hence can increase the decreasing speed of the tube voltage. This shortens the period D3 from time TLs (the time when the actually measured tube voltage starts to drop) according to Example 2 to time TL2 (the time when the actually measured tube voltage reaches the low tube voltage) as compared with the period D3 from time TLs to time TL1 according to Example 1.

An increase in actually measured grid voltage will be described in detail next. The grid control circuitry 83 immediately sets the target low tube current I2 to a set lower limit value and controls the grid voltage in accordance with tube current feedback based on the difference between the target tube current and the actually measured tube current immediately at time THs when an instruction to reduce the tube current is issued. The set lower limit value may be any value lower than the target low tube current I2, and it does not matter how much the value is lower than the target low tube current I2. For example, the set lower limit value may be, for example, slightly lower than the target low tube current I2, zero, or a value slightly higher than zero. In the increase period D1, the grid control circuitry 83 increases the actually measured grid voltage to a voltage V3 higher than the high grid voltage and then decreases the actually measured grid voltage to the high grid voltage by the high kV period D2. The actually measured grid voltage can be increased to the voltage V3 by, for example, amplifying the difference between the target tube current and the actually measured tube current. With a change in the grid voltage, the actually measured tube current decreases from the high tube current I1 to zero or a value near zero in the increase period D1 and then increases to the low tube current I2 by the high kV period D2. Rapidly increasing the grid voltage to the voltage V3 in the increase period D1 makes it possible to reduce the tube current to zero or a value near zero.

As the tube current decreases, the discharging of the electric charge accumulated in the tube voltage power supply circuitry 71 is suppressed. This makes it possible to increase the increasing speed of the tube voltage. Accordingly, the period D1 from time THs (the time when the actually measured tube voltage starts to increase) to time TH2 (the time when the actually measured tube voltage reaches the high tube voltage) according to Example 2 is shortened as compared with the period D1 from time THs to time TH1 according to Example 1.

According to Example 2, in the decrease period D3, increasing the tube current up to the upper limit tube current Ilim under grid control will promote the decreasing of the actually measured tube voltage. In the increase period D1, as the tube current is reduced to the set lower limit value under grid control, the increasing of the actually measured tube voltage is promoted. This makes it possible to shorten the decrease period D3 and the increase period D1 and prolong the low kV period D4 and the high kV period D2. The projection data acquired in the decrease period D3 and the increase period D1 cannot be used for image reconstruction, and only the projection data acquired in the low kV period D4 and the high kV period D2 can be used for image reconstruction. According to Example 2, shortening the decrease period D3 and the increase period D1 can improve the acquisition efficiency of projection data that can be used for image reconstruction.

The method of decreasing the grid voltage in the decrease period D3 is not limited to the above method. For example, the grid control circuitry 83 may decrease the grid voltage in accordance with a table (a gradient table hereinafter) recording the gradients of decreases in grid voltage. If the grid voltage is rapidly decreased, devices may be damaged. It is therefore preferable that gradients that avoid damage to devices are set in the gradient table. A gradient table is preferably prepared for each set tube voltage. For an increase in the grid voltage in the increase period D1, a gradient table generated for the increase may be used.

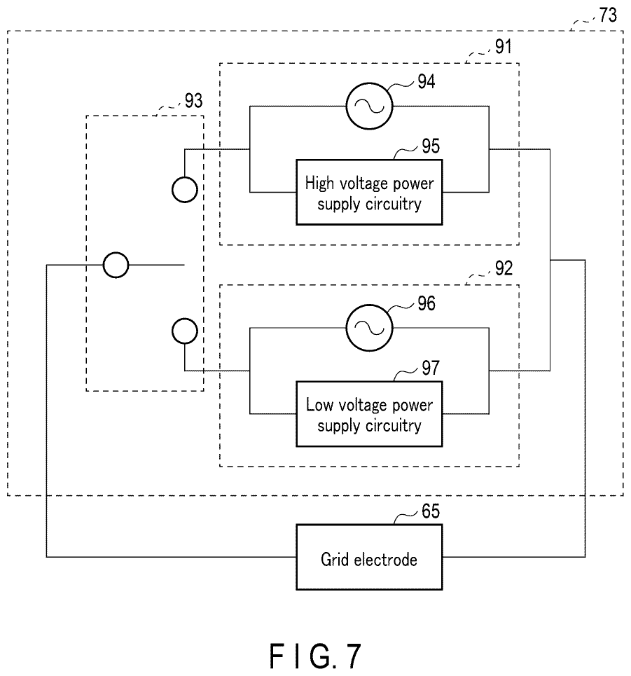

The following is a description about the arrangement of the grid power supply circuitry 73 according to Example 2 which is suitable for switching of the grid voltage between the low grid voltage and the high grid voltage. The grid power supply circuitry 73 has an electric storage element array that can switch between a parallel circuitry having electric storage elements connected in parallel and a series circuitry having electric storage elements connected in series. The grid power supply circuitry 73 operates as the parallel circuitry in the low kV period D4 and as the series circuitry in the high kV period D2. The grid power supply circuitry 73 will be described in detail below with reference to FIG. 7.

FIG. 7 shows an example of the arrangement of the grid power supply circuitry 73 according to Example 2. As shown in FIG. 7, the grid power supply circuitry 73 includes a circuitry 91, a circuitry 92, and a switch 93. The circuitry 91 and the circuitry 92 are connected to the grid electrode 65 via the switch 93. The circuitry 91 has a power supply 94 and a high voltage power supply circuitry 95. The power supply 94 has a power supply capacity.

The circuitry 92 has a power supply 96 and a low voltage power circuitry 97. The power supply 96 has a power supply capacity for the low grid voltage. The power supply capacity of the power supply 96 may be smaller than that of the power supply 94 for the high grid voltage. This makes it possible to reduce the costs of the power supply 94 and the power supply 96.

The switch 93 is a circuitry element that switches continuity to the grid electrode 65 between the circuitry 91 and the circuitry 92. The grid control circuitry 83 switches the switch 93. In response to an instruction to reduce the tube current (THs), the grid control circuitry 83 connects the switch 93 to the circuitry 91 to increase the grid voltage to the high voltage by using the circuitry 91 in the increase period D1 and holds the grid voltage at the high voltage in the high kV period D2. In response to an instruction to increase the tube current (TLs), the grid control circuitry 83 connects the switch 93 to the circuitry 92 to decrease the grid voltage to the low voltage by using the circuitry 92 in the decrease period D3 and hold the grid voltage at the low voltage in the low kV period D4. Switching between the circuitry 91 and the circuitry 92 in this manner can properly switch the grid voltage between the high voltage and the low voltage.

Referring to FIG. 7, although it is assumed that the grid power supply circuitry 73 is constituted by the circuitry 91 and the circuitry 92, the circuitry 91 and the circuitry 92 may be implemented by one circuitry that can switch between series connection and parallel connection of electric storage elements. Assume that the series-parallel connection grid power supply circuitry differs in only circuitry arrangement from the grid power supply circuitry 73 but has the same function.

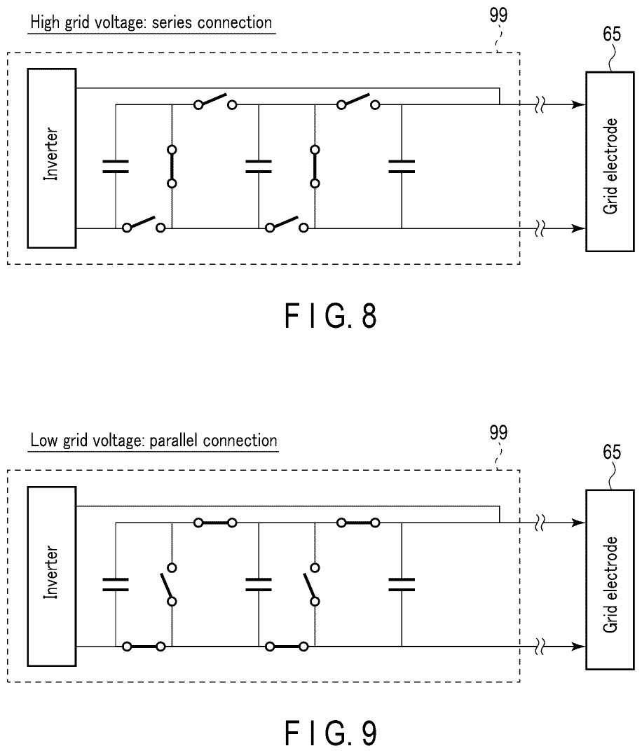

FIG. 8 is a schematic circuitry diagram of a series-parallel connection grid power supply circuitry 99, showing the connection relationship between switches forming a series circuitry at the time of applying the high grid voltage. FIG. 9 is a schematic circuitry diagram of the series-parallel connection grid power supply circuitry 99, showing the connection relationship between switches forming a parallel circuitry at the time of applying the low grid voltage. The inverters in FIGS. 8 and 9 correspond to the power supplies 94 and 96 in FIG. 7. As shown in FIGS. 8 and 9, the grid power supply circuitry 99 can switch between series connection and parallel connection by switching the switches. The grid power supply circuitry 99 allows a reduction in circuitry size as compared with the parallel circuitry 91 and the series circuitry 92 as separate circuits shown in FIG. 7. Note that the grid power supply circuitry 73 and 99 shown in FIGS. 7, 8, and 9 can also be used in Example 1.

### Example 3

The kV switching according to Examples 3 to 5 is combined with tube current modulation (AEC: Auto Exposure Control). In combining tube current modulation with kV switching, the X-ray control circuitry 85 controls the tube current control circuitry 84 to modulate the tube current in conjunction with the rotational angle of the X-ray tube 11 or the rotating frame 13 while controlling the tube voltage control circuitry 81 to alternately switch between the high tube voltage and the low tube voltage. As in Examples 3 to 5, the grid control circuitry 83 performs control to decrease the actually measured grid voltage to the low grid voltage and control to hold it exclusively within a decrease period and a low kV period so as to prevent the actually measured tube current from exceeding the upper limit tube current Ilim. In addition, as in Examples 3 to 5, the grid control circuitry 83 may perform control to decrease the actually measured grid voltage to the low grid voltage and control to hold it exclusively within a low kV period so as to prevent the actually measured tube current from exceeding the upper limit tube current Ilim. For example, “control to decrease the actually measured grid voltage to the low grid voltage” according to Examples 3 to 5 may be control to decrease the actually measured grid voltage from the high grid voltage to the low grid voltage as in Example 1 or may be control to increase the actually measured grid voltage from the voltage V2 to the low grid voltage after control to actively decrease the actually measured grid voltage from the high grid voltage to the voltage V2 lower than the low grid voltage.

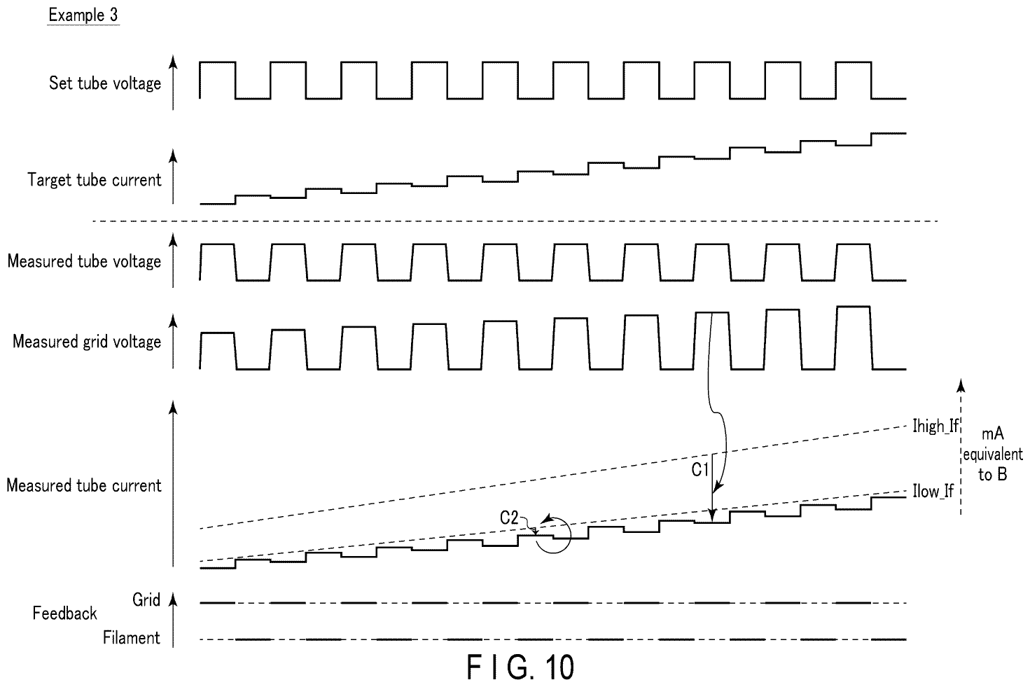

FIG. 10 is a timing chart of kV switching in Example 3. Unlike FIGS. 3 to 6, for the sake of descriptive convenience, FIG. 10 omits the illustration of an increase period, a high kV period, a decrease period, and a low kV period and also omits the illustration of the behaviors of actually measured grid voltages and actually measured tube currents in increase periods and decrease periods of tube voltages. In tube current modulation, two types of target tube currents, namely, a current for the high tube voltage and a current for the low tube voltage, are set. Each target tube current is set so as to increase stepwise with an increase in the body thickness of the subject P or X-ray transmission path length and decrease stepwise with a decrease in both thickness or X-ray transmission path length. As the target tube current is modulated, the If equivalent tube current Ihigh_If at the time of application of the high tube voltage and the If equivalent tube current Ilow_If at the time of application of the low tube voltage are modulated. FIG. 10 shows only a partial interval of the 360° angle range in which the target tube current gradually increases.

As the tube voltage is switched between the high voltage and the low voltage, the grid voltage is also switched between the high voltage and the low voltage. In Example 3, the low grid voltage is set to zero, and the high grid voltage is set to the grid voltage determined based on the target tube current and the allowable power at the current time.

As indicated by the lowermost row of FIG. 10, in the low kV period, the tube current control circuitry 84 instructs the filament control circuitry 82 to execute tube current feedback control. Upon reception of the instruction, the filament control circuitry 82 executes tube current feedback control based on the adjustment of a filament current in order to reduce the difference between the target tube current and the actually measured tube current corresponding to the low kV period. In the high kV period D2, the tube current control circuitry 84 instructs the grid control circuitry 83 to execute tube current feedback control. Upon reception of the instruction, the grid control circuitry 83 executes tube current feedback control based on the adjustment of a grid voltage in order to reduce the difference between the target tube current and the actually measured tube current corresponding to the high kV period D2.

According to Example 3, tube current feedback control based on the difference between a set tube current and an actually measured tube current is performed such that the filament control circuitry 82 executes feedback control based on a filament current in a low kV period, and the grid control circuitry 83 executes feedback control based on a grid voltage in a high kV period. Performing both tube current feedback control based on the adjustment of a filament current and tube current feedback control based on the adjustment of a grid voltage can reduce the consumption of the filament 62 as compared with the case where tube current feedback control based on the adjustment of a filament current is always executed.

### Example 4

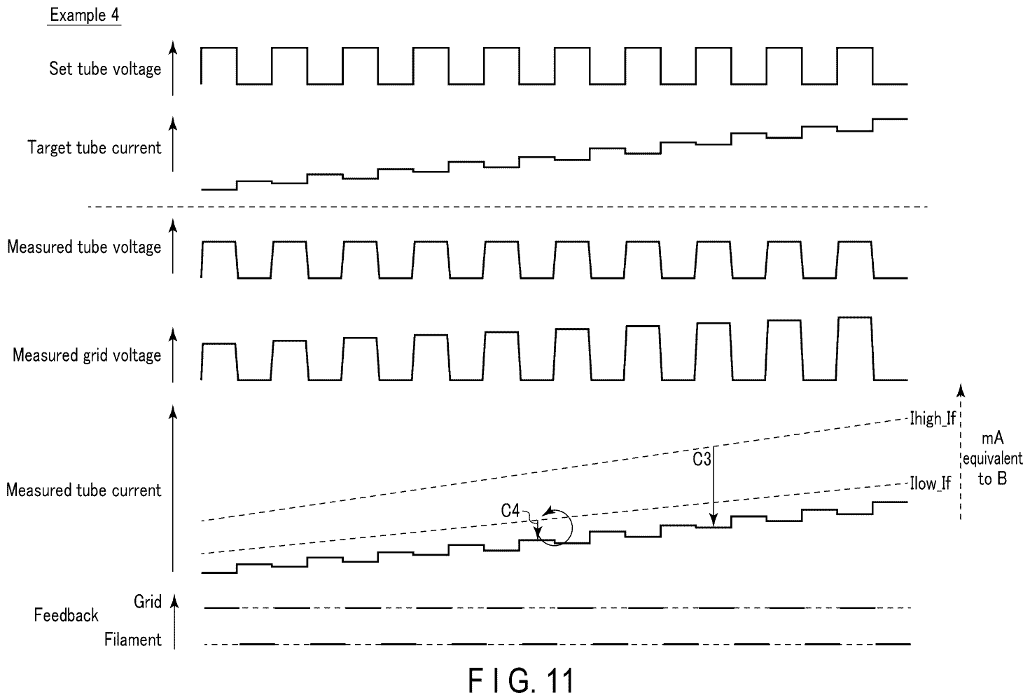

FIG. 11 is a timing chart of kV switching in Example 4. Similar to FIG. 10, FIG. 11 omits the illustration of an increase period, a high kV period, a decrease period, and a low kV period and also omits the illustration of the behaviors of actually measured grid voltages and actually measured tube currents in increase periods and decrease periods of tube voltages. As shown in FIG. 11, the low grid voltage according to Example 4 is set to a predetermined value higher than zero unlike in Example 3.

As shown in FIG. 11, the tube current control circuitry 84 instructs the filament control circuitry 82 to execute feedback control in a low kV period. Upon reception of the instruction, the filament control circuitry 82 executes feedback control based on a filament current in order to reduce the difference between the target tube current and the actually measured tube current corresponding to the low kV period. The tube current control circuitry 84 instructs the grid control circuitry 83 to execute feedback control in a high kV period. Upon reception of the instruction, the grid control circuitry 83 executes feedback control based on a grid voltage in order to reduce the difference between the target tube current and the actually measured tube current corresponding to the high kV period.

As described above, the low grid voltage is set to a predetermined value larger than zero. Therefore, according to Example 4, as in Example 2, it is possible to shorten decrease period 3 by temporally decreasing the actually measured grid voltage to the voltage V2 (see FIG. 6) lower than the low grid voltage in decrease period 3.

According to Example 3, the actually measured tube current makes a transition so as to approach the tube current Ilow_If as compared with Example 4. Accordingly, a suppression amount C1 (see FIG. 10) of tube current due to grid control in a high kV period is smaller than a suppression amount C3 (see FIG. 11) according to Example 4. In addition, a suppression amount C2 (see FIG. 10) of tube current due to grid control in a low kV period is smaller than a suppression amount C4 (see FIG. 11) according to Example 4. Therefore, Example 3 can reduce the consumption of the filament 62 as compared with Example 4.

### Example 5

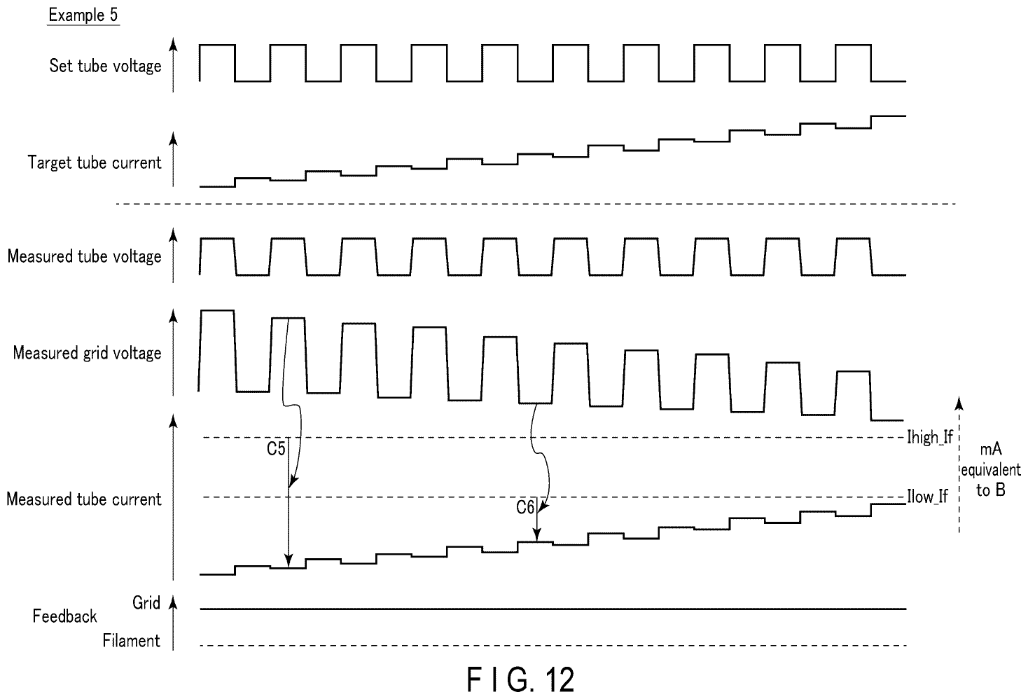

FIG. 12 is a timing chart of kV switching in Example 5. Similar to FIGS. 10 and 11, FIG. 12 omits the illustration of an increase period, a high kV period, a decrease period, and a low kV period and also omits the illustration of the behaviors of actually measured grid voltages and actually measured tube currents in increase periods and decrease periods of tube voltages.

Assume that Example 5 uses a constant If scheme as a filament current control scheme. In this case, as the grid control circuitry 83 performs tube current feedback control based on the adjustment of a grid voltage, the grid voltage changes in accordance with a target high tube current, and the high grid voltage changes in accordance with a target high tube current. In addition, due to the use of the constant If scheme for filament currents, the If equivalent tube current Ihigh_If at the time of application of the high tube voltage and the If equivalent tube current Ilow_If at the time of the low tube voltage each make a constant transition.

As shown in FIG. 12, in both a high kV period and a low kV period, the tube current control circuitry 84 instructs the filament control circuitry 82 to execute filament current control based on the constant If scheme and instructs the grid control circuitry 83 to execute feedback control based on a grid voltage. Upon reception of the instruction, the filament control circuitry 82 supplies a constant filament current to the filament 62 in the high kV period and the low kV period. The filament current is set to a current value within which the maximum power is not exceeded even when the maximum tube current corresponding to the low tube voltage flows. In a low kV period, the grid control circuitry 83 executes tube current feedback control based on the adjustment of the grid voltage in order to reduce the difference between the set high tube current and the actually measured tube current. In a high kV period, the grid control circuitry 83 executes a tube current feedback control based on the adjustment of the grid voltage in order to reduce the difference between the set low tube current and the actually measured tube current.

According to Example 5, unlike Examples 3 and 4, there is no need to execute tube current feedback control based on the adjustment of the filament current. Since the thermal inertia of the filament 62 is relatively large, the responsiveness of a tube current relative to a change in filament current is relatively low. Therefore, performing all tube current feedback control by tube current feedback control based on the adjustment of the grid voltage can modulate a tube current at a high speed.

The suppression amounts C1 and C3 (see FIGS. 10 and 11) of tube currents due to grid control in a high kV period according to Examples 3 and 4 are smaller than a suppression amount C5 (see FIG. 12) according to Example 5. In addition, the suppression amounts C2 and C4 (see FIGS. 10 and 11) of tube currents due to grid control in a low kV period are smaller than a suppression amount C6 (see FIG. 12) according to Example 5. Therefore, Examples 3 and 4 can reduce the consumption of the filament 62 and the grid electrode 65 as compared with Example 5.

### (Modification 1)

Although kV switching according to Examples 3 and 4 is combined with tube current modulation, the present embodiment is not limited to this. The tube current control circuitry 84 according to modification 1 is configured to combine kV switching according to Examples 3 and 4 with tube current non-modulation with a constant target high tube current and a constant target low tube current. In this case, in a low kV period, the filament control circuitry 82 executes feedback control based on a filament current in order to reduce the difference between a set high tube current and an actually measured tube current corresponding to the low kV period, whereas in a high kV period, the grid control circuitry 83 executes feedback control based on a grid voltage in order to reduce the difference between a set low tube current and an actually measured tube current corresponding to the high kV period.

The low grid voltage may be set to zero according to Example 3 or may be set to a predetermined value higher than zero according to Example 4.

### (Modification 2)

The X-ray tube control system 100 according to the present embodiment includes the X-ray tube 11, the tube voltage power supply circuitry 71, the filament heating circuitry 72, the grid power supply circuitry 73, and the control circuitry 74. However, the X-ray tube control system 100 may not include the X-ray tube 11, the tube voltage power supply circuitry 71, the filament heating circuitry 72, and the grid power supply circuitry 73. In other words, the X-ray tube control system 100 may include only the control circuitry 74. In this case, the X-ray tube control system 100 may be provided on the console 40 of the X-ray computed tomography imaging apparatus 1 or may be provided on a server computer connected to the X-ray computed tomography imaging apparatus 1 via a network. The control circuitry 74 can control the tube voltage power supply circuitry 71, the filament heating circuitry 72, and the grid power supply circuitry 73 via a network.

In addition, the X-ray tube control system 100 may not include the X-ray tube 11, the tube voltage power supply circuitry 71, the filament heating circuitry 72, and the grid power supply circuitry 73. In other words, the X-ray tube control system 100 may include only the X-ray high voltage device 14.

According to at least one embodiment described above, it is possible to reduce the difference between the X-ray dose at the time of application of the high tube voltage and the X-ray dose at the time of application of the low tube voltage.

The term “processor” used in the above description means a circuit such as a CPU, GPU, ASIC (Application Specific Integrated Circuit), SPLD (Simple Programmable Logic Device), CPLD (Complex Programmable Logic Device), or FPGA (Field Programmable Logic Device). The processor reads out a program stored in a storage circuit and executes the program to implement the corresponding function. Note that a program may be directly incorporated in a circuit of the processor instead of being saved in a storage circuit. In this case, the processor reads out the program incorporated in the circuit and executes the program to implement the corresponding function. In contrast to this, if the processor is, for example, an ASIC, the corresponding function is directly incorporated as a logic circuit in the circuit of the processor instead of saving a program in the storage circuit. Note that each processor according to the present embodiment may be formed as one processor by combining a plurality of independent circuits to implement the corresponding function as well as being formed as a single circuit. In addition, a plurality of constituent elements in FIGS. 1 and 2 may be integrated into one processor to implement the corresponding function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray tube control system comprising:
a tube voltage control circuitry configured to alternately switch a tube voltage applied between a cathode that emits an electron and an anode that generates an X-ray upon reception of the electron from the cathode between a first tube voltage and a second tube voltage higher than the first tube voltage; and
a grid control circuitry configured to alternately switch a grid voltage applied between the cathode and a grid electrode that controls an electron propagating from the cathode to the anode between a first grid voltage and a second grid voltage higher than the first grid voltage,
wherein the grid control circuitry performs control to decrease a grid voltage to the first grid voltage and control to hold the first grid voltage exclusively within a first transition period in which a tube voltage makes a transition from the second tube voltage to the first tube voltage and a first hold period in which the tube voltage is held at the first tube voltage so as to prevent a tube current from exceeding an upper limit tube current based on allowable power.

2. The X-ray control system according to claim 1, wherein the grid control circuitry starts to decrease a grid voltage in response to a decrease in tube voltage to a reference value between the first tube voltage and the second tube voltage or the first tube voltage.

3. The X-ray control system according to claim 1, wherein the grid control circuitry decreases a grid voltage so as to cause a tube current not less than a first target tube current at time of application of the first tube voltage to flow up to the upper limit tube current in the first transition period.

4. The X-ray tube control system according to claim 3, wherein the grid control circuitry decreases a grid voltage to a voltage lower than the first grid voltage in the first transition period and then increases the grid voltage to the first grid voltage by the first hold period.

5. The X-ray tube control system according to claim 3, further comprising a grid power supply circuitry configured to apply a grid voltage to the grid electrode,
wherein the grid power supply circuitry includes an electric storage element array configured to switch between a parallel circuitry having electric storage elements connected in parallel and a series circuitry having electric storage elements connected in series and operates as the parallel circuitry in the first hold period and as the series circuitry in a second hold period in which a tube voltage is held at the second tube voltage.

6. The X-ray tube control system according to claim 1, wherein the grid control circuitry increases a grid voltage to reduce a tube current to a set lower limit value in a second transition period in which a tube voltage makes a transition from the first tube voltage to the second tube voltage.

7. The X-ray tube control system according to claim 6, wherein the grid control circuitry increases a grid voltage to a voltage higher than the second grid voltage in the second transition period and then decreases the grid voltage to the second grid voltage by a second hold period in which the second tube voltage is held at the second tube voltage.

8. The X-ray tube control system according to claim 6, further comprising a grid power supply circuitry configured to apply a grid voltage to the grid electrode,
wherein the grid power supply circuitry includes an electric storage element array configured to switch between a parallel circuitry having electric storage elements connected in parallel and a series circuitry having electric storage elements connected in series and operates as the parallel circuitry in the first hold period and as the series circuitry in a second hold period in which a tube voltage is held at the second tube voltage.

9. The X-ray tube control system according to claim 1, further comprising a filament control circuitry configured to control a filament current supplied to a filament of the cathode,
wherein the filament control circuitry executes feedback control based on a filament current in a first hold period in order to reduce a difference between a first target tube current and an actually measured tube current corresponding to the first hold period, and
the grid control circuitry executes feedback control based on a grid voltage in a second hold period, in which a tube voltage is held at the second tube voltage, in order to reduce a difference between a second target tube current and an actually measured tube current corresponding to the second hold period.

10. The X-ray tube control system according to claim 9, wherein the first grid voltage is zero.

11. The X-ray tube control system according to claim 9, wherein the first grid voltage is a constant value higher than zero.

12. The X-ray tube control system according to claim 1, further comprising a filament control circuitry configured to control a predetermined heating current that heats a filament of the cathode,
wherein the grid control circuitry executes feedback control based on a grid voltage in the first hold period in order to reduce a difference between a first target tube current and an actually measured tube current corresponding to the first hold period and executes feedback control based on a grid voltage in a second hold period, in which a tube voltage is held at the second tube voltage, in order to reduce a difference between a second target tube current and an actually measured tube current corresponding to the second hold period.

13. The X-ray tube control system according to claim 1, wherein the grid control circuitry controls a grid voltage to cause a modulation target tube current to follow an actually measured tube current.

14. The X-ray tube control system according to claim 1, further comprising:
an X-ray tube including the cathode, the anode, and the grid electrode;
a tube voltage power supply circuitry configured to apply a tube voltage between the cathode and the anode under the control of the tube voltage control circuitry; and
a grid power supply circuitry configured to apply a grid voltage between the cathode and the grid electrode under the control of the grid control circuitry.

15. An X-ray tube control system comprising:
a tube voltage control circuitry configured to alternately switch a tube voltage applied between a cathode that emits an electron and an anode that generates an X-ray upon reception of the electron from the cathode between a first tube voltage and a second tube voltage higher than the first tube voltage; and
a grid control circuitry configured to alternately switch a grid voltage applied between the cathode and a grid electrode that controls the electron propagating from the cathode to the anode between a first grid voltage and a second grid voltage higher than the first grid voltage,
wherein the grid control circuitry performs control to hold a grid voltage at the first grid voltage exclusively within a first hold period in which a tube voltage is held at the first tube voltage.

16. An X-ray computed tomography imaging apparatus comprising:
an X-ray tube including a cathode that emits an electron, an anode that generates an X-ray upon reception of the electron from the cathode, and a grid electrode that controls the electron propagating from the cathode to the anode;
an X-ray detector configured to detect an X-ray generated from the anode and transmitted through a subject;
a data acquisition circuitry configured to acquire projection data via the X-ray detector;
a tube voltage control circuitry configured to alternately switch a tube voltage applied between the cathode and the anode between a first tube voltage and a second tube voltage higher than the first tube voltage; and
a grid control circuitry configured to alternately switch a grid voltage applied between the cathode and the grid electrode between a first grid voltage and a second grid voltage higher than the first grid voltage,
wherein the grid control circuitry performs control to decrease a grid voltage to the first grid voltage and control to hold the first grid voltage exclusively within a first transition period in which a tube voltage makes a transition from the second tube voltage to the first tube voltage and a first hold period in which the tube voltage is held at the first tube voltage so as to avoid a tube current from exceeding an upper limit tube current based on allowable power.
